(19) United States
(12) Patent Application Publication
(10) Pub. No.: US 2026/0290584 A1
(43) Pub. Date: Sep. 24, 2026

(54) EMOTION-BASED DATA ANALYSIS SYSTEM FOR USER EMOTION MONITORING AND OPERATING METHOD THEREOF

(71) Applicants: HYUNDAI MOTOR COMPANY, SEOUL (KR); KIA CORPORATION, SEOUL (KR)

(72) Inventors: Yu Jung Chae, Hwaseong-si (KR); Hyun Jin Kang, Hwaseong-si (KR)

(73) Assignees: HYUNDAI MOTOR COMPANY, SEOUL (KR); KIA CORPORATION, SEOUL (KR)

(21) Appl. No.: 19/312,240

(22) Filed: Aug. 27, 2025

(30) Foreign Application Priority Data
Mar. 24, 2025 (KR) 10-2025-0037541

(51) Int. Cl. G16H 50/20; G06T 7/73; G06V 40/16; G10L 25/24; G10L 25/63; G16H 10/20; G16H 40/20

(52) U.S. Cl. CPC G16H 50/20; G06T 7/73; G16H 10/20; G16H 40/20; G06T 2207/10024; G06T 2207/30201; G06V 40/174; G10L 25/24; G10L 25/63

(57) ABSTRACT

An emotion-based data analysis system for user emotion monitoring includes data storage that stores a predefined question set and a healthy person's emotion pattern, a scenario controller that sequentially executes questions and controls an answer acquisition start time point and an answer acquisition end time point of a user, a preprocessing device that obtains pieces of data indicating an emotion of the user to generate preprocessed data, an individual emotion extraction device that predicts valence and arousal, a final emotion determination device that analyzes predicted values of the valence and the arousal to and primarily extracts the calculated value as a final representative emotion, a user emotion pattern analysis device that compares the primarily extracted final representative emotion with the healthy person's emotion pattern and secondarily extracts a final representative emotion, and an abnormal data storage server device that receives and stores the secondarily extracted final representative emotion.

### CROSS-REFERENCE TO RELATED APPLICATION(S)

This application claims the benefit of priority to Korean Patent Application No. 10-2025-0037541, filed in the Korean Intellectual Property Office on Mar. 24, 2025, the entire contents of which are incorporated herein by reference.

### TECHNICAL FIELD

The present disclosure relates to an emotion-based data analysis system for user emotion monitoring and an operating method thereof, and more particularly, relates to technologies for obtaining data, such as an utterance (e.g., voice) and/or text of a patient, in real time in daily life via a domestic robot and extracting and providing abnormal data to a specialist to minimize a data monitoring time of the specialist.

### BACKGROUND

Various psychological counseling services for people who suffer psychologically and emotionally have been provided. In most psychological counseling services, accurately predicting the emotion of a counselee may be a useful measure of service quality. Nevertheless, it may be difficult to accurately predict the emotion of the counselee using (e.g., only) an utterance (e.g., sentence) due to unique characteristics of various languages, such as the Korean language. Furthermore, because most psychological counseling services derive (e.g., only) the result of analyzing the emotion of the counselee as psychological counseling data, a specialist may find it challenging to monitor a vast amount of raw data related to the counselee. Thus, a method for more accurately predicting the emotion of the counselee and extracting (e.g., only) abnormal data related to the emotion of the counselee may be useful for the specialist to have a more focused set of data for consideration, such as providing the specialist with (e.g., only) abnormal data to consider.

### SUMMARY

The present disclosure has been made to address the above-mentioned problems.

An aspect of the present disclosure provides an emotion-based data analysis system for user emotion monitoring for synthesizing multimodal data, such as a pose, an expression, a voice, or an utterance (e.g., sentence) of a user via conversion between a robot (e.g., a robotic vacuum cleaner) and a user to predict the emotion of the user, determining data in which the degree of a difference with an emotion pattern of a healthy person (e.g., a healthy person may be considered a person having various attributes such as absence of diagnosed diseases, no ongoing treatment, stable vital signs, and good mental and physical function) among the predicted emotions of the user is large (e.g., based on the design of the system) and may be considered as abnormal data (e.g., the concept of “abnormal” is determined based on the system designer's criteria). The system further is configured to upload the abnormal data to a server for monitoring by a specialist. The user may be a patient or person targeted for emotion monitoring and may be seeking and/or may require diagnosis and treatment by a specialist. The specialist may be a medical doctor. Depending on the predicted emotion, the specialist may make a diagnosis or determine, provide, and/or offer treatment or a treatment plan to the user. The emotions considered in an “emotion pattern” can vary depending on the system design and implementation.

Another aspect of the present disclosure provides an operating method of an emotion-based data analysis system for user emotion monitoring for synthesizing multimodal data, such as a pose, an expression, a voice, or an utterance (e.g., sentence) of a user via conversion between a robot and a user to predict the emotion of the user, determining data in which the degree of a difference with an emotion pattern of a healthy person among the predicted emotions of the user is large as abnormal data, and uploading the abnormal data to a server for monitoring by a specialist.

The present disclosure is not limited to the aforementioned problems, and other technical problems, not mentioned herein, may be understood from the following description to which the present disclosure pertains.

According to an aspect of the present disclosure, an emotion-based data analysis system for user emotion monitoring may include data storage that stores a predefined question set and a healthy person's emotion pattern corresponding to the predefined question set. The system may include a scenario controller that sequentially executes questions constituting the predefined question set and controls an answer acquisition start time (e.g., point) and an answer acquisition end time (e.g., point) of a user for the questions, a preprocessing device that obtains data (e.g., pieces of data) indicating an emotion of the user to generate preprocessed data, in response to a user answer acquisition start signal received from the scenario controller, and an individual emotion extraction device that predicts valence (e.g., pleasure) and arousal for (e.g., each of the pieces of) data indicating the emotion of the user based on the preprocessed data received from the preprocessing device. The system also may include a final emotion determination device that analyzes predicted values of the valence and the arousal for (e.g., each piece) of the data indicating the emotion of the user, the predicted values being received from the individual emotion extraction device, in a two-dimensional (2D) space to calculate an average emotion value of an area with highest density and primarily extract the calculated value as a final representative emotion, and a user emotion pattern analysis device that compares the primarily extracted final representative emotion with the healthy person's emotion pattern stored in the data storage and secondarily extracts a final representative emotion in which a difference with the healthy person's emotion pattern is greater than or equal to a predetermined value. The system further may include an abnormal data storage server device that receives and stores the secondarily extracted final representative emotion from the user emotion pattern analysis device. The predetermined value may be determined by the system designer.

In an example embodiment, the data (e.g., pieces of data) indicating the emotion of the user may include one or more of pose data, expression data, voice data, or text data of the user.

In an example embodiment, the predefined question set stored in the data storage may be plural in number. The scenario controller may select one question set among the plurality of predefined question sets stored in the data storage and may sequentially deliver questions constituting the selected question set to the user.

In an example embodiment, the predefined question set may include an Nth question and an (N+1)th question subsequent to the Nth question. The scenario controller may execute the Nth question and may execute the (N+1)th question, in response to that there is no text conversion corresponding to the Nth question during a predetermined time. The predetermined time may be determined by the system designer.

In an example embodiment, the emotion-based data analysis system may further include a camera and a microphone that obtain and provide the data (e.g., pieces of data) indicating the emotion of the user to the preprocessing device, in response to that the preprocessing device receives the user answer acquisition start signal.

In an example embodiment, the preprocessing device may perform RGB conversion of the data (e.g., pieces of data) indicating the emotion of the user, the data (e.g., pieces of data) being received from the camera, on a frame-by-frame basis and may extract landmarks of a pose and an expression of the user from RGB data converted on the frame-by-frame basis to generate the preprocessed data.

In an example embodiment, the extracted landmark of the pose of the user may include at least one pair of a joint ID of the user and a 2D coordinate value corresponding to the joint ID of the user.

In an example embodiment, the extracted landmark of the expression of the user may include at least one pair of a feature point ID of a face of the user and a 2D coordinate value corresponding to the feature point ID of the face of the user.

In an example embodiment, the preprocessing device may store the data (e.g., pieces of data) indicating the emotion of the user, the data (e.g., pieces of data) being received from the microphone, in a buffer and may extract a voice feature of voice data stored in the buffer via a Mel-frequency cepstral coefficient (MFCC) to generate the preprocessed data.

In an example embodiment, the preprocessing device may store the data (e.g., pieces of data) indicating the emotion of the user, the data (e.g., pieces of data) being received from the microphone, in a buffer, may convert voice data stored in the buffer into text data, and may convert the converted text data into a computer-readable vector via a word embedding model to generate the preprocessed data.

According to another aspect of the present disclosure, an operating method of an emotion-based data analysis system for user emotion monitoring is provided. The method may include storing, by data storage, a predefined question set and a healthy person's emotion pattern corresponding to the predefined question set, sequentially executing, by a scenario controller, questions constituting the predefined question set, controlling, by the scenario controller, an answer acquisition start time and an answer acquisition end time of a user for the questions, receiving, by a preprocessing device, a user answer acquisition start signal from the scenario controller, obtaining, by the preprocessing device, data (e.g., pieces of data) indicating an emotion of the user to generate preprocessed data, in response to receiving the user answer acquisition start signal, receiving, by an individual emotion extraction device, the preprocessed data from the preprocessing device, predicting, by the individual emotion extraction device, valence and arousal for (e.g., each piece of) the data indicating the emotion of the user, receiving, by a final emotion determination device, predicted values of the valence and the arousal for (e.g., each piece of) the data indicating the emotion of the user from the individual emotion extraction device, analyzing, by the final emotion determination device, the predicted values of the valence and the arousal for each (e.g., piece of) the data indicating the emotion of the user, in a two-dimensional (2D) space to calculate an average emotion value of an area with highest density, primarily extracting, by the final emotion determination device, the calculated value as a final representative emotion, comparing, by a user emotion pattern analysis device, the primarily extracted final representative emotion with the healthy person's emotion pattern stored in the data storage and secondarily extracting, by the user emotion pattern analysis device, a final representative emotion in which a difference with the healthy person's emotion pattern is greater than or equal to a predetermined value, and receiving and storing, by an abnormal data storage server device, the secondarily extracted final representative emotion from the user emotion pattern analysis device.

In an example embodiment, the data (e.g., pieces of data) indicating the emotion of the user may include one or more of pose data, expression data, voice data, or text data of the user.

In an example embodiment, the predefined question set stored in the data storage may be plural in number. The sequential executing of the questions constituting the predefined question set by the scenario controller may include selecting, by the scenario controller, one question set among the plurality of predefined question sets and sequentially delivering, by the scenario controller, questions constituting the selected question set to the user.

In an example embodiment, the predefined question set may include an Nth question and an (N+1)th question subsequent to the Nth question. The sequential executing of the questions constituting the predefined question set by the scenario controller may include executing, by the scenario controller, the Nth question and executing, by the scenario controller, the (N+1)th question, in response to that there is no text conversion corresponding to the Nth question during a predetermined time.

In an example embodiment, the operating method may further include obtaining and providing, by a camera and a microphone, the data (e.g., pieces of data) indicating the emotion of the user to the preprocessing device, in response to that the preprocessing device receives the user answer acquisition start signal.

In an example embodiment, the generating of the preprocessed data by the preprocessing device may include performing RGB conversion of the data (e.g., pieces of data) indicating the emotion of the user, the data (e.g., pieces of data) being received from the camera, on a frame-by-frame basis and extracting landmarks of a pose and an expression of the user from RGB data converted on the frame-by-frame basis to generate the preprocessed data. The landmarks may be one or more feature points on the contour of the user's face.

In an example embodiment, the extracted landmark of the pose of the user may include at least one pair of a joint ID of the user and a 2D coordinate value corresponding to the joint ID of the user.

In an example embodiment, the extracted landmark of the expression of the user may include at least one pair of a feature point ID of a face of the user and a 2D coordinate value corresponding to the feature point ID of the face of the user. n an example embodiment, the generating of the preprocessed data by the preprocessing device may include storing the data (e.g., pieces of data) indicating the emotion of the user, the data (e.g., pieces of data) being received from the microphone, in a buffer and extracting a voice feature of voice data stored in the buffer via a Mel-frequency cepstral coefficient (MFCC) to generate the preprocessed data.

In an example embodiment, the generating of the preprocessed data by the preprocessing device may include storing the data (e.g., pieces of data) indicating the emotion of the user, the data (e.g., pieces of data) being received from the microphone, in a buffer; converting voice data stored in the buffer into text data, and converting the converted text data into a computer-readable vector via a word embedding model to generate the preprocessed data.

### BRIEF DESCRIPTION OF THE DRAWINGS

The above and other objects and features of the present disclosure may be more apparent from the following detailed description taken in conjunction with the accompanying drawings:

FIG. 1 is a drawing illustrating a configuration of a user emotion monitoring system according to an example embodiment of the present disclosure;

FIG. 2 is a drawing illustrating a configuration of an emotion-based data analysis system for user emotion monitoring according to an example embodiment of the present disclosure;

FIG. 3 is a flowchart for describing an operating method of an emotion-based data analysis system for user emotion monitoring according to an example embodiment of the present disclosure;

FIG. 4 is a drawing for describing an operating method of an emotion-based data analysis system for user emotion monitoring according to an example embodiment of the present disclosure;

FIG. 5 is a drawing illustrating a predefined question set stored in data storage according to an example embodiment of the present disclosure;

FIG. 6 is a drawing for describing an operation of preprocessing pose data and expression data in a preprocessing device according to an example embodiment of the present disclosure;

FIGS. 7 and 8 are drawings for describing pieces of preprocessed data generated by a preprocessing device according to an example embodiment of the present disclosure;

FIG. 9 is a drawing for describing an operation of preprocessing voice data and text data in a preprocessing device according to an example embodiment of the present disclosure;

FIG. 10 is a drawing for describing an individual emotion extraction device according to an example embodiment of the present disclosure;

FIG. 11 is a drawing for describing valence and arousal of the emotion of a user according to an example embodiment of the present disclosure;

FIG. 12 is a drawing for describing an operation of predicting valence and arousal in an individual emotion extraction device according to an example embodiment of the present disclosure;

FIG. 13 is a drawing for describing an operation of predicting valence and arousal in a pose emotion prediction modeling device of an individual emotion extraction device according to an example embodiment of the present disclosure;

FIG. 14 is a drawing for describing an operation of extracting a frequency feature value in an MFCC voice feature extraction device according to an example embodiment of the present disclosure;

FIG. 15 is a drawing for describing an operation of extracting a word vector value in a word embedding modeling device according to an example embodiment of the present disclosure;

FIGS. 16, 17, 18, and 19 are drawings for describing a method for primarily extracting a final emotion in a final emotion determination device according to an example embodiment of the present disclosure;

FIG. 20 is a drawing for describing a healthy person's emotion pattern model stored in data storage according to an example embodiment of the present disclosure; and

FIGS. 21 and 22 are drawings for describing a method for determining abnormal data using a healthy person's emotion pattern model stored in data storage and secondarily extracting a final emotion of a user in a user emotion pattern analysis device according to an example embodiment of the present disclosure.

FIG. 23 is a drawing for describing an example embodiment of a computing system for use with or in an emotion-based data analysis system for user emotion monitoring according to an example embodiment of the present disclosure.

### DETAILED DESCRIPTION

Hereinafter, some embodiments of the present disclosure will be described in detail with reference to the exemplary drawings. In adding the reference numerals to the components of each drawing, it should be noted that the the same or similar components may be designated by the same numerals, even when they are displayed on other drawings. Further, in describing the embodiment of the present disclosure, a detailed description of various features or functions may not be included to avoid obscuring the gist of the present disclosure.

In describing the components of the example embodiment of the present disclosure, terms such as first, second, “A”, “B”, (a), (b), and the like may be used. These terms are used to distinguish one component from another component, but do not limit the corresponding components irrespective of the order or priority of the corresponding components. Furthermore, unless otherwise provided (e.g., defined), terms including technical and scientific terms used herein have the same meaning as generally understood by those skilled in the art to which the present disclosure pertains. Such terms as those provided (e.g., defined) in a generally used dictionary are to be interpreted as having meanings the same as the contextual meanings in the relevant field, and are not to be interpreted as having ideal or excessively formal meanings unless provided (e.g., defined) as having such in the present application.

When a controller, component, device, element, part, unit, module, or the like of the present disclosure is described as having a purpose or performing an operation, function, or the like, the controller, component, device, element, part, unit, or module should be considered herein as being “configured to” meet that purpose or perform that operation or function. Each controller, component, device, element, part, unit, module, and the like may separately embody or include a processor and/or a memory, such as a non-transitory computer-readable medium, as part of the system and/or apparatus.

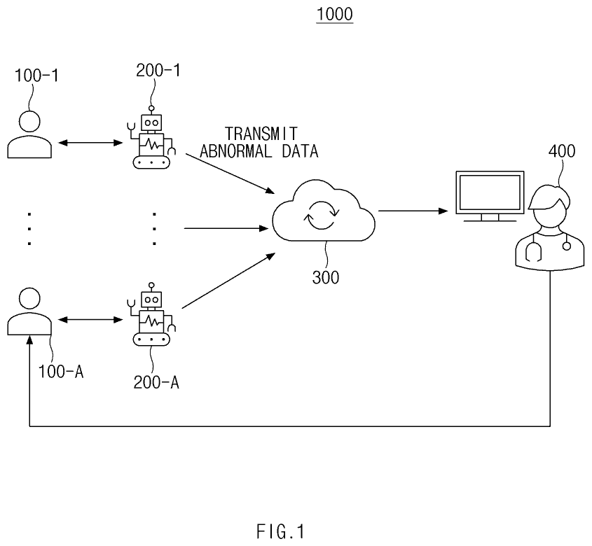

Hereinafter, embodiments of the present disclosure will be described in detail with reference to FIGS. 1 to 22.

FIG. 1 is a drawing illustrating a configuration of a user emotion monitoring system according to an example embodiment of the present disclosure.

Referring to FIG. 1, a user emotion monitoring system 1000 may include a plurality of users 100-1 to 100-A, a plurality of robots 200-1 to 200-A, a monitoring server 300, and a specialist 400 (here, A is a natural number of 1 or more). Each of the plurality of users 100-1 to 100-A may be a user who is targeted for emotion monitoring. The plurality of robots 200-1 to 200-A may correspond to the plurality of users 100-1 to 100-A, respectively. Hereinafter, a description of an example of the robot 200-1 and the user 100-1, which are paired with each other, is provided. However, the description below may also be applied to the robots 200-2 to 200-A and the users 100-2 to 100-A, which are paired with each other, in the same manner.

The robot 200-1 and the user 100-1 corresponding to the robot 200-1 may transmit and receive an utterance (e.g., voice) and/or text to have a conversation with each other. The robot 200-1 may be a domestic robot, which may be present together in a daily life (e.g., space) of the user 100-1. The robot 200-1 may sequentially deliver questions constituting a predefined question set to the user 100-1. When the user 100-1 takes a specific pose or an expression in response to the delivered questions or utters a specific voice or text, the robot 200-1 may obtain data (e.g., pieces of data) indicating the emotion of the user 100-1 in real time. The robot 200-1 may process the obtained data (e.g., pieces of data), may extract data capable of helping the specialist 400 to diagnose and treat the user 100-1 using (e.g., based on) the processed data (e.g., pieces of data), and may provide the monitoring server 300 with the extracted data. At this time, the data capable of helping the specialist 400 to diagnose and treat the user 100-1 may be abnormal data, which may be data indicating the emotion of the user 100-1, which has a large degree of difference upon comparison with an emotion pattern of a healthy person among emotion patterns of the user 100-1. The large degree of difference may be part of the design of the system 1000.

The monitoring server 300 may store the abnormal data delivered from the robot 200-1 and may provide the specialist 400 with the abnormal data, thus minimizing time taken for the specialist 400 to monitor the emotion of the user 100-1 and analyze data indicating the emotion of the user 100-1.

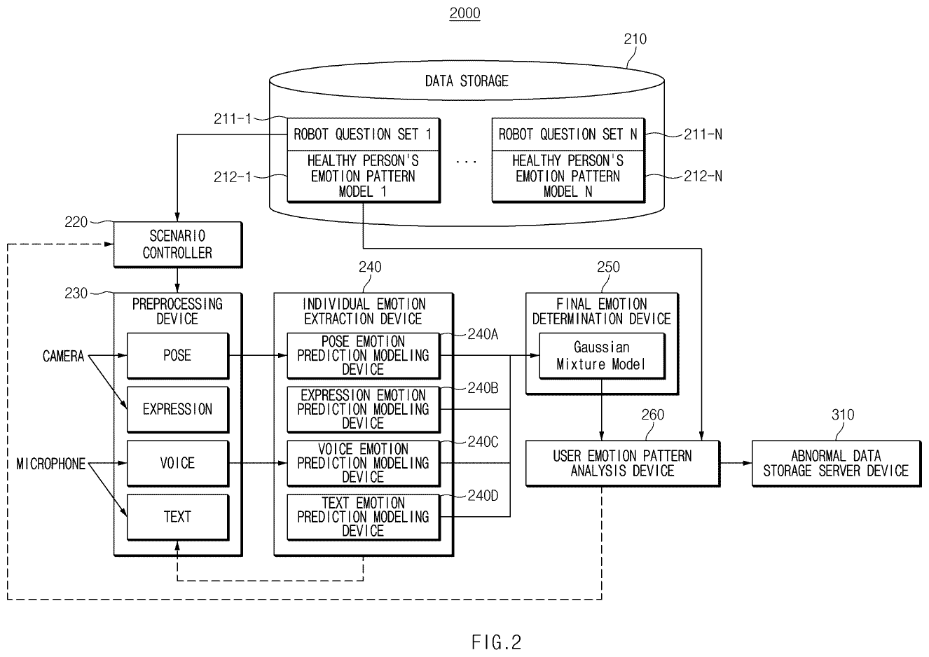

FIG. 2 is a drawing illustrating a configuration of an emotion-based data analysis system for user emotion monitoring according to an example embodiment of the present disclosure.

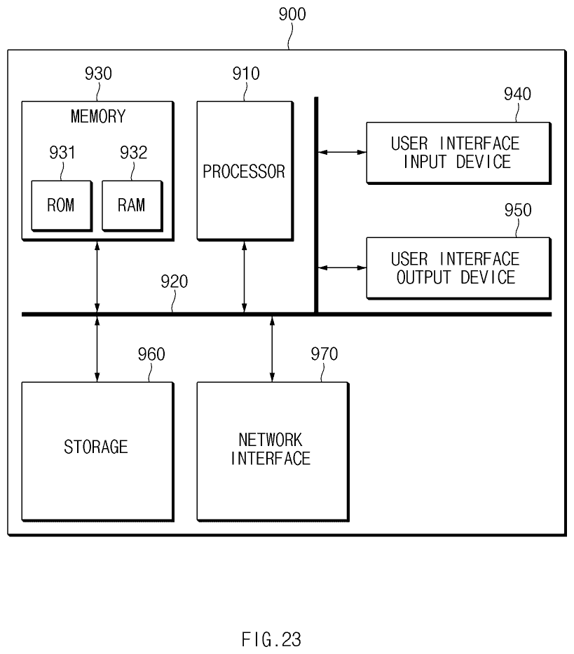

Referring to FIG. 2, an emotion-based data analysis system 2000 for user emotion monitoring may include data storage 210, a scenario controller 220, a preprocessing device 230, an individual emotion extraction device 240, a final emotion determination device 250, a user emotion pattern analysis device 260, and an abnormal data storage server device 310. The scenario controller 220, the preprocessing device 230, the individual emotion extraction device 240, the final emotion determination device 250, the user emotion pattern analysis device 260, and the abnormal data storage server device 310 may each include a storage, a memory, and a processor as disclosed in FIG. 23 and described hereinbelow. In an example embodiment, each device 230, 240, 250, and/or 260 may include a communication circuit.

The data storage 210 may include a plurality of predefined question sets 211-1 to 211-N and a plurality of healthy person's emotion pattern models 212-1 to 212-N (here, N is a natural number of 2 or more). The predefined questions of the question sets 211-1 to 211-N may be configured (e.g., set) to assess the user's emotional state. For example, questions may include “How are you feeling?”, “Have you been stressed lately?”, and/or “How was your day?”

The predefined question sets 211-1 to 211-N and the healthy person's emotion pattern models 212-1 to 212-N may correspond to each other, respectively. For example, each of the predefined question sets 211-1 to 211-N may include at least one of predefined question list. Furthermore, each of the healthy person's emotion pattern models 212-1 to 212-N may include a healthy person's emotion pattern. At this time, the healthy person's emotion pattern included in the healthy person's emotion pattern model may correspond to a question list included in a predefined question set corresponding to the healthy person's emotion pattern model.

In some example embodiments, the predefined question set 211-1 may be stored as a JSON, XML, or TXT file. A storage path of the healthy person's emotion pattern model 212-1 corresponding to the predefined question set 211-1 may be stored together in the file. Thus, the healthy person's emotion pattern model 212-1 corresponding to the predefined question set 211-1 may be (e.g., explicitly) displayed.

The scenario controller 220 may select one of the plurality of predefined question sets 211-1 to 211-N stored in the data storage 210. Thereafter, the scenario controller 220 may (e.g., sequentially) execute a question list constituting the selected predefined question set and may query the user 100-1. Thereafter, the scenario controller 220 may control a time point for starting to obtain an answer of the user 100-1 to a question of the robot 200-1 and a time point for ending the answer of the user 100-1. For example, the scenario controller 220 may transmit a user answer acquisition start signal to the preprocessing device 230 to control the preprocessing device 230 to start user answer acquisition. Furthermore, the scenario controller 220 may transmit a user answer acquisition end signal to the preprocessing device 230 to control the preprocessing device 230 to end the user answer acquisition.

The preprocessing device 230 may obtain data (e.g., pieces of data) indicating the emotion of the user 100-1 via a camera and a microphone in response to the user answer acquisition start signal received from the scenario controller 220, after the question of the robot 200-1 controlled by the scenario controller 220 ends, and may perform preprocessing for the obtained data (e.g., pieces of data). Thereafter, the preprocessing device 230 may end the preprocessing in response to the user answer acquisition end signal received from the scenario controller 220. As such, the preprocessing device 230 may perform preprocessing for the data (e.g., pieces of data) indicating the emotion of the user 100-1 to generate preprocessed data. In some example embodiments, the data (e.g., pieces of data) indicating the emotion of the user 100-1, which are obtained using the camera and the microphone by the preprocessing device 230, may include one or more of pose data, expression data, voice data, or text data of the user 100-1.

The individual emotion extraction device 240 may receive the preprocessed data from the preprocessing device 230 and may analyze the received pieces of preprocessed data on a time-by-time basis (e.g., on a second-by-second basis) to analyze an emotion for the respective data (e.g., pieces of data). For example, the individual emotion extraction device 240 may predict a valence value and an arousal value for (e.g., each piece of) the data indicating the emotion of the user 100-1 using a pose emotion prediction modeling device 240A, an expression emotion prediction modeling device 240B, a voice emotion prediction modeling device 240C, and a text emotion prediction modeling device 240D based on the received preprocessed data and may analyze the emotion for (e.g., each piece of) the data indicating the emotion of the user 100-1 using the predicted valence value and the predicted arousal value.

The final emotion determination device 250 may receive predicted values of the valence and the arousal for (e.g., each piece of) the data indicating the emotion of the user 100-1 from the individual emotion extraction device 240. Furthermore, the final emotion determination device 250 may analyze the received predicted values in a 2D space and may primarily extract an average emotion value in an area with the highest density as a final representative emotion.

The user emotion pattern analysis device 260 may receive the primarily extracted final representative emotion from the final emotion determination device 250. Furthermore, the user emotion pattern analysis device 260 may compare the received primarily extracted final representative emotion with the healthy person's emotion pattern model 212-1 stored in the data storage 210. At this time, the healthy person's emotion pattern model 212-1, which is targeted for comparison, may correspond to the predefined question set 211-1 selected by the scenario controller 220.

The user emotion pattern analysis device 260 may compare the final representative emotion primarily extracted by the final emotion determination device 250 with the healthy person's emotion pattern model 212-1 corresponding to the predefined question set 211-1 selected by the scenario controller 220 and may secondarily extract a final representative emotion in which a difference with the emotion pattern of the healthy person's emotion pattern model 212-1 among the primarily extracted final representative emotions is greater than or equal to a predetermined value.

The abnormal data storage server device 310 may receive the secondarily extracted final representative emotion from the user emotion pattern analysis device 260 and may store the received final representative emotion.

In some example embodiments, the data storage 210, the scenario controller 220, the preprocessing device 230, the individual emotion extraction device 240, the final emotion determination device 250, and the user emotion pattern analysis device 260 may be included in a robot 200-1. The other robots 200-2 to 200-A constituting a user emotion monitoring system 1000 may include each of the configurations (e.g., configurations of the data storage 210, the scenario controller 220, the preprocessing device 230, the individual emotion extraction device 240, the final emotion determination device 250, and/or the user emotion pattern analysis device 260). Furthermore, the abnormal data storage server device 310 may be the same configuration as a monitoring server 300 of FIG. 1.

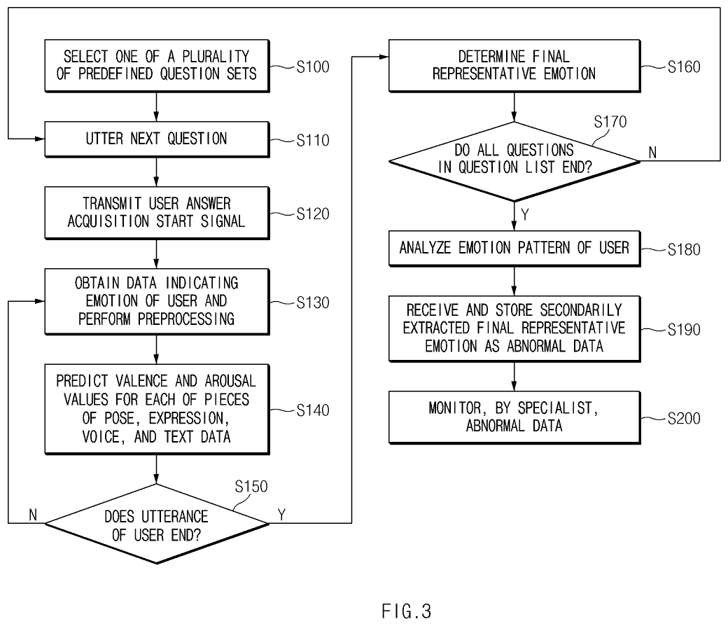

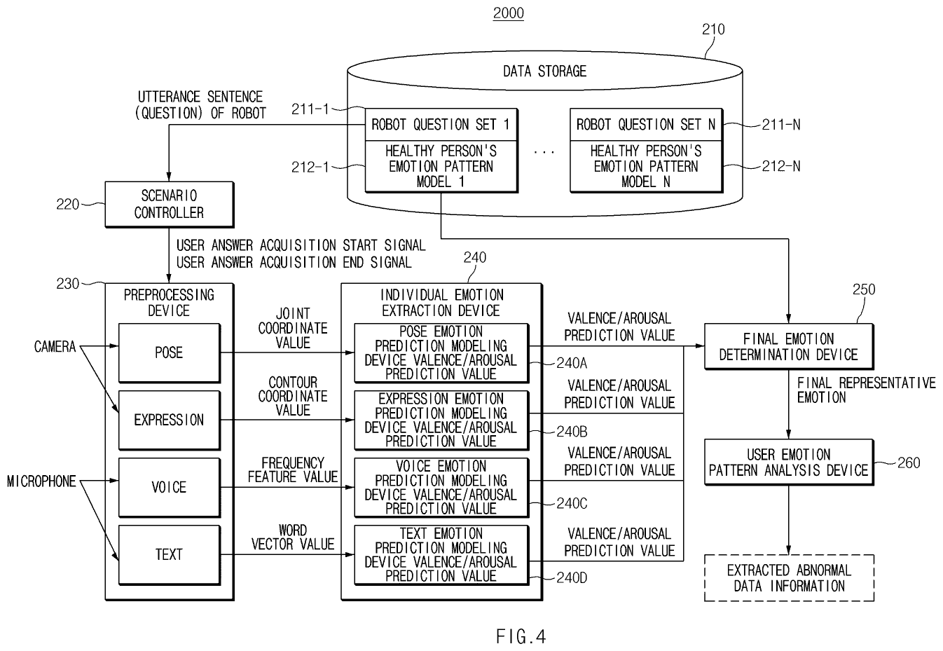

FIG. 3 is a flowchart for describing an operating method of an emotion-based data analysis system for user emotion monitoring according to an example embodiment of the present disclosure. FIG. 4 is a drawing for describing an operating method of an emotion-based data analysis system for user emotion monitoring according to an example embodiment of the present disclosure.

Hereinafter, a description will be given of the operating method of the emotion-based data analysis system for user emotion monitoring according to an example embodiment of the present disclosure with reference to FIGS. 3 and 4.

First, in S100, a scenario controller 220 may select one of a plurality of predefined question sets 211-1 to 211-N stored in data storage 210. At this time, the scenario controller 220 may select any one of the plurality of predefined question sets 211-1 to 211-N stored in the data storage 210 in a time specified by a user 100-1. However, an example embodiment is not limited thereto. The scenario controller 220 may select a specified question set among the plurality of predefined question sets 211-1 to 211-N stored in the data storage 210 on a day specified by a setting of the user 100-1.

Next, in S110, the scenario controller 220 may (e.g., sequentially) execute a question list constituting the selected predefined question set and may articulate (e.g., utter) a question to the user 100-1. At this time, the scenario controller 220 may convert a question sentence constituting the selected predefined question set stored in the form of text into a voice and may deliver the converted voice to the user 100-1.

Next, in S120, the scenario controller 220 may transmit a user answer acquisition start signal to a preprocessing device 230 to request the preprocessing device 230 to start user answer acquisition. Next, in S130, the preprocessing device 230 may obtain data indicating the emotion of the user 100-1 via a camera and a microphone in response to the user answer acquisition start signal received from the scenario controller 220 and may perform preprocessing for the obtained data. The preprocessing device 230 may perform preprocessing for the data indicating the emotion of the user 100-1 to generate preprocessed data.

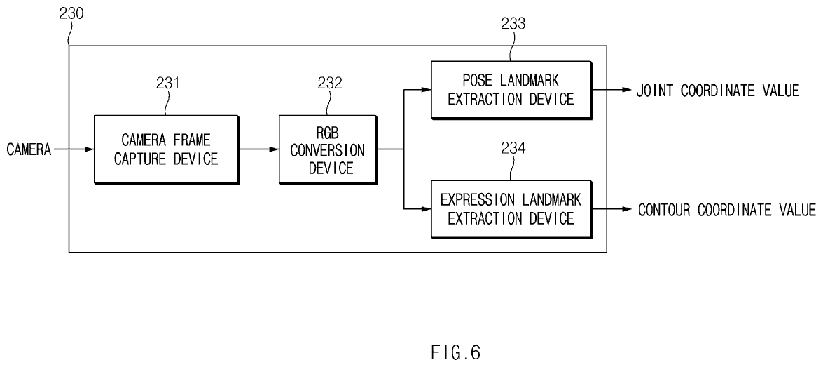

Data (e.g., pieces of data) generated by preprocessing pose data and expression data of the user 100-1, which are received from the camera, by the preprocessing device 230 may be a joint coordinate value and a contour coordinate value, respectively. Furthermore, data (e.g., pieces of data) generated by preprocessing voice data and text data of the user 100-1, which are received from the microphone, by the preprocessing device 230 may be a frequency feature value and a word vector value, respectively. A description hereinbelow of the preprocessed data generated by the preprocessing device 230 by preprocessing the data (e.g., pieces of data) indicating the emotion of the user 100-1, which are received from the camera and the microphone, is provided with reference to FIGS. 6 to 9.

In some example embodiments, an emotion-based data analysis system 2000 for user emotion monitoring may include the camera and the microphone. The camera and the microphone may obtain and provide the data (e.g., pieces of data) indicating the emotion of the user 100-1 to the preprocessing device 230, in response to that the preprocessing device 230 receives the user answer acquisition start signal from the scenario controller 220. At this time, the data obtained by the camera may be pose data and expression data of the user 100-1, and the data obtained by the microphone may be voice data and text data of the user 100-1.

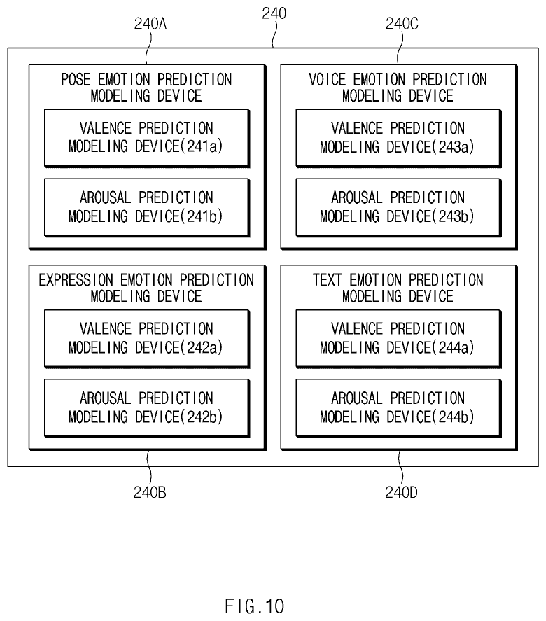

Next, in S140, the individual emotion extraction device 240 may analyze the pieces of preprocessed data received from the preprocessing device 230 and may predict valence and arousal values for each (e.g., piece of) pose, expression, voice, and text data received from the preprocessing device 230. In some example embodiments, the individual emotion extraction device 240 may configure a learning model suitable for a characteristic of each data to predict valence and arousal values for (e.g., each piece of) data to extract an individual emotion from the pose, expression, voice, and text data. The individual emotion extraction device 240 may use a deep learning model, and a pre-trained model may be applied. There is no limitation on the type of learning model used by the device 240, but examples of such models may include Long Short-Term Memory (LSTM), Bidirectional Long Short-Term Memory (BiLSTM), and/or Convolutional Neural Networks (CNN), which process time-series data in a supervised learning manner. These models receive preprocessed data as input and output values such as valence and arousal. The learning model may be optionally applied by the system designer. A description hereinbelow of the operation of predicting the valence and arousal values for (e.g., each piece of) data received from the preprocessing device 230 by the individual emotion extraction device 240 is provided with reference to FIGS. 10 to 15.

Next, in S150, the scenario controller 220 may determine whether the utterance of the user 100-1 for the question constituting the predefined question set ends. For example, the scenario controller 220 may recognize a time point when an answer of the user 100-1 to each question constituting the selected predefined question set ends and may determine whether the utterance of the user 100-1 ends. In detail, assuming that the predefined question set includes an Mth question (here, M is a natural number of 2 or more), the scenario controller 220 may execute the Mth question and may determine that the answer of the user 100-1 to the Mth question is completed, when there is no process of receiving a voice answer of the user 100-1 to the Mth question and converting the voice answer into text (hereinafter referred to as “text conversion”) during a predetermined time (hereinafter referred to as an “answer waiting time”). Thereafter, the scenario controller 220 may execute an (M+1)th question. At this time, the answer waiting time may be adjusted according to an age group of the user 100-1. The scenario controller 220 may manage an utterance (e.g., sentence) turn (e.g., a “speaking turn” or a “taking a turn”) of a robot 200-1.

When it is determined that the utterance of the user 100-1 to the question constituting the predefined question set does not end (S150-N), in S130, the scenario controller 220 may allow the preprocessing device 230 to continue obtaining data indicating the emotion of the user 100-1 via the camera and the microphone for the question and generate preprocessed data. Meanwhile, when it is determined that the utterance of the user 100-1 for the question constituting the predefined question set ends (S150—Y), in S160, a final emotion determination device 250 may determine a final representative emotion. For example, the final emotion determination device 250 may receive predicted values of valence and arousal for (e.g., each piece of) the data indicating the emotion of the user 100-1 from the individual emotion extraction device 240 and may analyze the received predicted values in a 2D space to primarily extract an average emotion value in an area with the highest density as the final representative emotion. “A high density” or the “highest density” may refer to an area within the 2D space (or 2D emotion space) where the concentration of the received predicted values is greater than in other areas.

Next, in S170, the scenario controller 220 may determine whether S110 to S160 for all the questions in the question list constituting the selected question set among the plurality of predefined question sets 211-1 to 211-N stored in the data storage 210 end. When it is determined that S110 to S160 for all the questions in the question list constituting the selected question set do not end (S170-N), in S110, the scenario controller 220 may utter a next question to the user 100-1.

For example, assuming that the predefined question set includes an Mth question and an (M+1)th question subsequent to the Mth question, in S110, the scenario controller 220 may determine that the answer of the user 100-1 to the Mth question is completed and may execute the (M+1)th question, when there is no operation of receiving the voice answer of the user 100-1 to the Mth question and converting the voice answer into text during an answer waiting time.

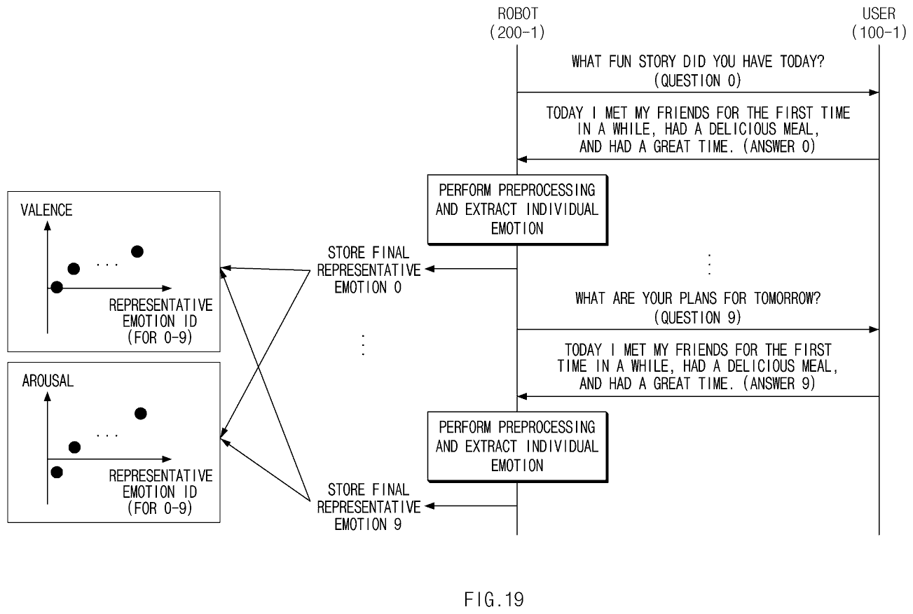

Meanwhile, when the scenario controller 220 determines that S110 to S160 for (e.g., all) the questions in the question list constituting the selected question set end (S170—Y), in S180, a user emotion pattern analysis device 260 may compare the final emotion primarily extracted by the final emotion determination device 250 with a healthy person's emotion pattern and may secondarily extract a final representative emotion to analyze an emotion pattern of the user 100-1. A description hereinbelow of the operation of comparing the primarily extracted final emotion with the healthy person's emotion pattern and secondarily extracting the final representative emotion in the user emotion pattern analysis device 260 is provided with reference to FIGS. 19 to 22.

Next, in S190, an abnormal data storage server device 310 may receive and store the secondarily extracted final representative emotion as abnormal data from the user emotion pattern analysis device 260. In S200, a specialist 400 may monitor the abnormal data stored in the abnormal data storage server device 310. The specialist 400 may evaluate changes in the condition of the user 100-1 by monitoring abnormal data, and may provide diagnosis and treatment to the user based on the results of such monitoring.

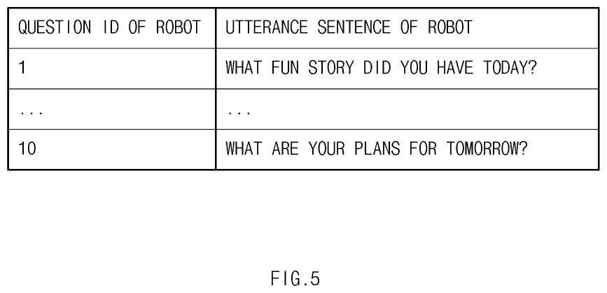

FIG. 5 is a drawing illustrating a predefined question set stored in data storage according to an example embodiment of the present disclosure.

Hereinafter, a description herein of an example of a predefined question set 211-1 among a plurality of predefined question sets 211-1 to 211-N included in data storage 210 is provided. However, the description below is also applicable to the remaining predefined question sets 211-2 to 211-N. Questions included in the predefined question set 211-1 may be composed in the form of a sentence and may be composed of around 10 sentences to observe a change in emotion pattern of a user 100-1 for monitoring an emotion state of the user 100-1. In some example embodiments, questions included in the predefined question set 211-1 to guide the user 100-1 to utter an answer may be in the form of an open question, rather than ending the answer of the user 100-1 in the form of a short answer.

FIG. 6 is a drawing for describing an operation of preprocessing pose data and expression data in a preprocessing device according to an example embodiment of the present disclosure.

Referring to FIG. 6, a preprocessing device 230 may include a camera frame capture device 231, an RGB (e.g., Red, Green, Blue) conversion device 232, a pose landmark extraction device 233, and an expression landmark extraction device 234. The camera frame capture device 231 may receive external data via a camera, while a user 100-1 gives an answer. In detail, the camera frame capture device 231 may capture a video image in which the user 100-1 gives an answer for each frame. The camera frame capture device 231 may transmit a raw image on a frame-by-frame basis to the RGB conversion device 232.

The RGB conversion device 232 may perform RGB conversion on the frame-by-frame basis using an OpenCV library for the raw image on the frame-by-frame basis, which is received from the camera frame capture device 231. The RGB conversion device 232 may transmit the converted RGB data on the frame-by-frame basis to the pose landmark extraction device 233 and the expression landmark extraction device 234. The pose landmark extraction device 233 may extract a pose landmark from the converted RGB data on the frame-by-frame basis. The expression landmark extraction device 234 may extract an expression landmark from the converted RGB data on the frame-by-frame basis. In some example embodiments, the pose landmark extraction device 233 and the expression landmark extraction device 234 may extract a pose coordinate value of the user 100-1 and an expression coordinate value of the user 100-1 from the converted RGB data on the frame-by-frame basis using a mediapipe library, respectively.

In some example embodiments, the pose landmark extracted by the pose landmark extraction device 233 may indicate a 2D coordinate value of a pose, which may extract a coordinate value in units of P joints of the user 100-1 (here, P is a natural number of 1 or more). At this time, an example of the joint of the user 100-1 may be a shoulder, an elbow, a wrist, a finger, a pelvis, or the like. The pose landmark extracted by the pose landmark extraction device 233 may include at least one pair of a joint ID of the user 100-1 and a 2D coordinate value corresponding to the joint ID.

For example, when the pose landmark extraction device 233 extracts a 2D coordinate value corresponding to a left wrist of the user 100-1 as (16, 20) and extracts a 2D coordinate value corresponding to a left shoulder of the user 100-1 as (52, 16), the joint coordinate value extracted by the pose landmark extraction device 233 may have a list form as follows (here, for example, the joint ID of the left wrist of the user 100-1 is 15 and the joint ID of the left shoulder is 11).

### Joint Coordinate Value

15 (left Wrist): (16, 20), 11 (left Shoulder): (52, 16), . . .

In some example embodiments, the expression landmark extracted by the expression landmark extraction device 234 may indicate a 2D coordinate value of an expression. The expression landmark extraction device 234 may extract a 2D coordinate value from a feature point of a face (e.g., eyebrows, eyes, a nose, a mouth, or the like) contour of the user 100-1 using a mediapipe face mesh. Like the pose landmark extracted by the pose landmark extraction device 233, the expression landmark extracted by the expression landmark extraction device 234 may include at least one pair of a feature point ID of the user 100-1 and a 2D coordinate value corresponding to the feature point ID.

For example, when the expression landmark extraction device 234 extracts a 2D coordinate value corresponding to the eyebrows of the user 100-1 as (15, 19) and extracts a 2D coordinate value corresponding to the nose of the user 100-1 as (53, 17), the contour coordinate value extracted by the expression landmark extraction device 234 may have a list form as follows (here, for example, the ID of an eyebrow contour of the user 100-1 is 16 and the joint ID of the nose contour is 10).

### Contour Coordinate Value

16 (eyebrow contour): (15, 19), 10 (nose contour): (53, 17), . . .

As such, the joint coordinate value and the contour coordinate value respectively generated by the pose landmark extraction device 233 and the expression landmark extraction device 234 of the preprocessing device 230 may be included in preprocessed data generated by the preprocessing device 230.

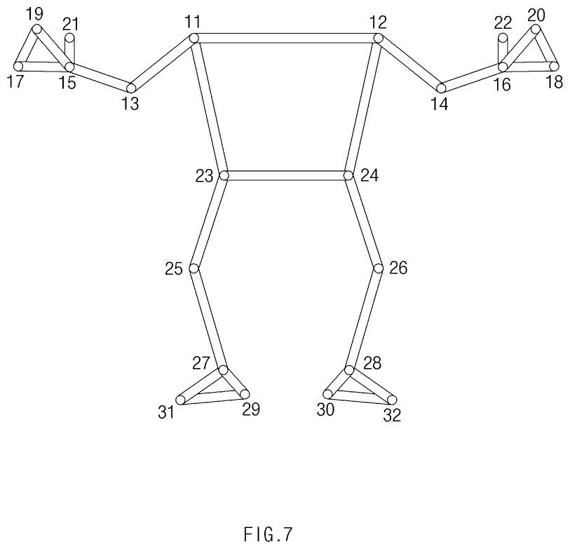

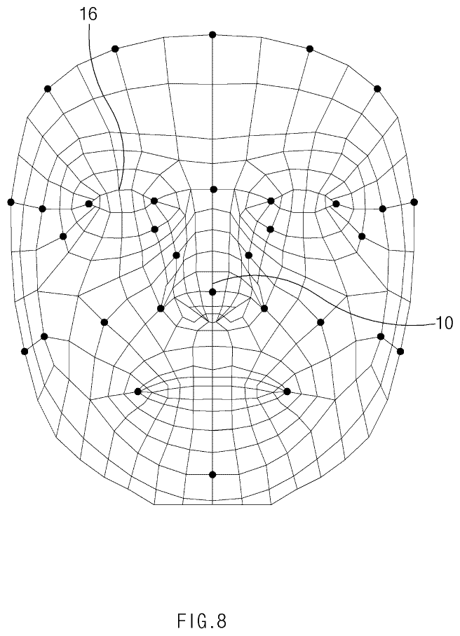

FIGS. 7 and 8 are drawings for describing pieces of preprocessed data generated by a preprocessing device according to an example embodiment of the present disclosure.

FIG. 7 is an exemplary drawing illustrating a joint ID which corresponds for each joint of a user 100-1 for each joint of the user 100-1 for a pose landmark extraction device 233 to extract a pose landmark including a joint coordinate value. Referring to FIG. 7, the joint ID may be matched for each joint of the user 100-1. For example, a joint ID of a left wrist of the user 100-1 may be 15, and a joint ID of a right wrist of the user 100-1 may be 16. Furthermore, a joint ID of a left shoulder of the user 100-1 may be 11, and a joint ID of a right wrist of the user 100-1 may be 12. Furthermore, a joint ID of a left ankle of the user 100-1 may be 27, and a joint ID of a right ankle of the user 100-1 may be 28. The joint ID of the user 100-1 shown in FIG. 7 is exemplified. According to an example embodiment, a detailed numerical value of the joint ID which corresponds to each joint of the user 100-1 may vary.

Next, FIG. 8 is an exemplary drawing illustrating a mediapipe face mesh for an expression landmark extraction device 234 to extract an expression landmark including a contour coordinate value. Referring to FIG. 8, a feature point ID may be matched for each face contour of the user 100-1. For example, a feature point ID of a left eyebrow of the user 100-1 may be 16, and a feature point ID of a nose of the user 100-1 may be 10.

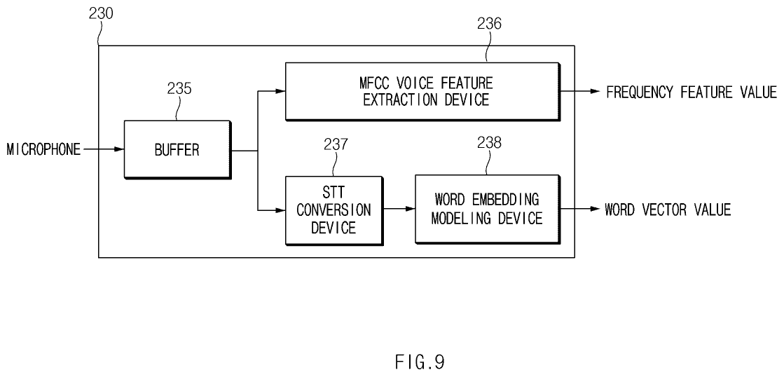

FIG. 9 is a drawing for describing an operation of preprocessing voice data and text data in a preprocessing device according to an example embodiment of the present disclosure.

Referring to FIG. 9, a preprocessing device 230 may include a buffer 235, a Mel-frequency cepstral coefficient (MFCC) voice feature extraction device 236, a speech to text (STT) conversion device 237, and a word embedding modeling device 238. The buffer 235 may store voice data of a user 100-1 (e.g., in real time) via a microphone, while the user 100-1 gives an answer. The preprocessing device 230 may extract a voice feature and an utterance (e.g., sentence) of the user 100-1 using the voice data of the user 100-1, which is accumulated in the buffer 235.

The buffer 235 may transmit the accumulated voice data of the user 100-1 to the MFCC voice feature extraction device 236 and the STT conversion device 237. The MFCC voice feature extraction device 236 may extract an MFCC feature from the voice data of the user 100-1 using a librosa package to generate a frequency feature value. MFCC is a technique for analyzing a spectrum of voice data and extracting a feature of the voice data.

The STT conversion device 237 may convert the voice data of the user 100-1, which is in the form of an utterance (e.g., sentence), into a text form to analyze the voice data. In some example embodiments, the STT conversion device 237 may be implemented with Google STT, which may convert voice raw data of the user 100-1, which is stored in the buffer 235, into text data. The STT conversion device 237 may transmit the text data to the word embedding modeling device 238.

The word embedding modeling device 238 may convert the converted text data into a computer-readable vector using a word embedding model to generate a word vector value. As such, the frequency feature value and the word vector value, respectively generated by the MFCC voice feature extraction device 236 and the word embedding modeling device 238 of the preprocessing device 230, may be included in preprocessed data generated by the preprocessing device 230.

FIG. 10 is a drawing for describing an individual emotion extraction device according to an example embodiment of the present disclosure.

Referring to FIG. 10, an individual emotion extraction device 240 may include a learning model suitable for a characteristic of each (e.g., piece of) data to extract an individual emotion of a user 100-1 from a joint coordinate value, a contour coordinate value, a frequency feature value, and a word vector value generated by a preprocessing device 230. For example, the individual emotion extraction device 240 may include a pose emotion prediction modeling device 240A, an expression emotion prediction modeling device 240B, a voice emotion prediction modeling device 240C, and a text emotion prediction modeling device 240D. Each model is discussed herein. The pose emotion prediction modeling device 240A may extract an individual emotion of the user 100-1 from a joint coordinate value, which is data about a pose of the user 100-1. The expression emotion prediction modeling device 240B may extract an individual emotion of the user 100-1 from a contour coordinate value, which is data about an expression of the user 100-1. The voice emotion prediction modeling device 240C may extract an individual emotion of the user 100-1 from a frequency feature value which is data about a voice of the user 100-1. The text emotion prediction modeling device 240D may extract an individual emotion of the user 100-1 from a word vector value, which is data about an utterance (e.g., sentence) of the user 100-1.

The pose emotion prediction modeling device 240A, the expression emotion prediction modeling device 240B, the voice emotion prediction modeling device 240C, and the text emotion prediction modeling device 240D may include valence prediction modeling devices 241a to 244a and arousal prediction modeling devices 241b to 244b. Hereinbelow, the valence prediction modeling device 241a and the arousal prediction modeling device 241b included in the pose emotion prediction modeling device 240A is described. However, the description below is also applicable to the descriptions of the other valence prediction modeling devices 242a, 243a, and 244a, and the other arousal prediction modeling devices 242b, 243b, and 244b in the same or similar manner.

The valence prediction modeling device 241a may predict valence of the emotion of the user 100-1, which corresponds to a joint coordinate value received from the preprocessing device 230. The arousal prediction modeling device 241b may predict arousal of the emotion of the user 100-1, which corresponds to a contour coordinate value received from the preprocessing device 230. At this time, the valence prediction modeling device 241a and the arousal prediction modeling device 241b may predict the valence and the arousal of the user 100-1 as a value between −1 and +1, respectively.

As such, each of the pose emotion prediction modeling device 240A, the expression emotion prediction modeling device 240B, the voice emotion prediction modeling device 240C, and the text emotion prediction modeling device 240D may be composed of 2 types of models to predict valence and arousal of the emotion of the user 100-1, which are included in preprocessed data including data indicating the emotion of the user 100-1. The configuration of the learning model included in the individual emotion extraction device 240 may be obtained from a (e.g., public) site, such as AI Hub.

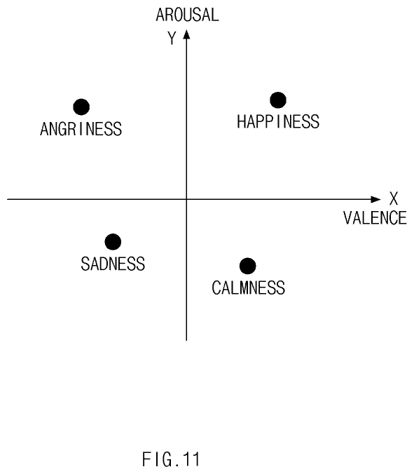

FIG. 11 is a drawing for describing valence and arousal of the emotion of a user according to an example embodiment of the present disclosure.

Referring to FIG. 11, an emotion-based data analysis system 2000 for user emotion monitoring may apply a 2D emotion model rather than a discontinuous emotion (e.g., sadness, happiness, angriness (e.g., anger), or the like) model to more precisely analyze an emotion pattern of a user 100-1. According to an example embodiment, the 2D emotion model of Russell may be applied to some embodiments of the present disclosure.

In the 2D emotion model shown in FIG. 11, the emotion of the user 100-1 may be (e.g., continuously) present in a 2D space. The X-axis may be composed of valence, and the Y-axis may be composed of arousal. At this time, the valence may indicate a positive disposition in the direction of the X-axis and may indicate a negative disposition in the direction opposite to the X-axis. Furthermore, the arousal may be an index indicating a (e.g., kind of) strength (or energy), which may indicate high strength in the direction of the Y-axis and may indicate low strength in the direction opposite to the Y-axis.

In some example embodiments, each of a valence value and an arousal value may be limited to a minimum value of −1 and a maximum value of +1 to normalize a valence prediction value and an arousal prediction value for a set of (e.g., plurality of pieces) of data indicating the emotion of the user 100-1.

For example, an emotion with both high valence and high arousal may correspond to the emotion of happiness among discontinuous emotions, an emotion with high valence and low arousal may correspond to the emotion of calmness among the discontinuous emotions, an emotion with low valence and high arousal may correspond to the emotion of angriness (e.g., anger) among the discontinuous emotions, and an emotion with both low valence and low arousal may correspond to the emotion of sadness among the discontinuous emotions.

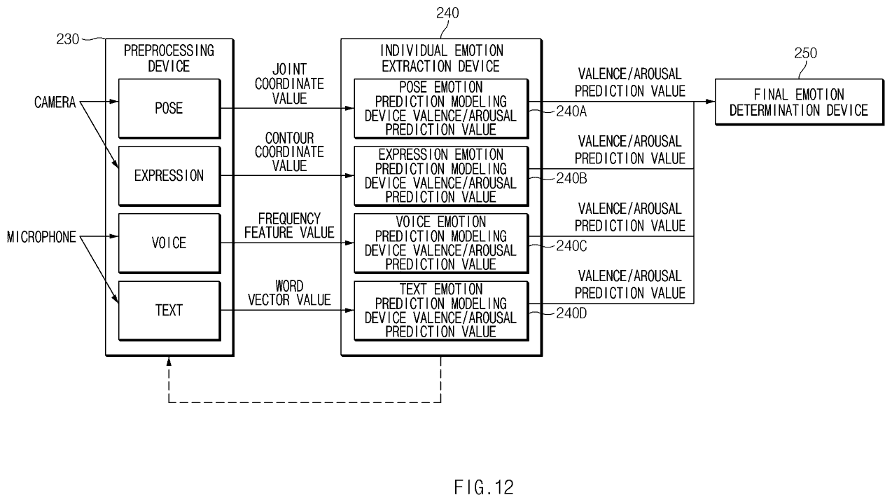

FIG. 12 is a drawing for describing an operation of predicting valence and arousal in an individual emotion extraction device according to an example embodiment of the present disclosure.

Referring to FIG. 12, each of modeling devices 240A to 240D of an individual emotion extraction device 240 may store a value accumulated by predicting an individual emotion of a user 100-1 at the same prediction period or different prediction periods during an answer time of the user 100-1 to a question of a robot 200-1. For example, when the answer time of the user 100-1 is 30 seconds and the prediction period of each of the pose emotion prediction modeling device 240A and the expression emotion prediction modeling device 240B is 3 seconds, each of the pose emotion prediction modeling device 240A and the expression emotion prediction modeling device 240B may generate a valence prediction value and an arousal prediction value for 10 individual emotions. At this time, the valence prediction value and the arousal prediction value for each individual emotion may be paired with each other. Thus, in this example, each of the pose emotion prediction modeling device 240A and the expression emotion prediction modeling device 240B may generate total 10 pairs of predicted values.

For example, when the answer time of the user 100-1 is 30 seconds and the prediction period of each of the voice emotion prediction modeling device 240C and the text emotion prediction modeling device 240D is 5 seconds, each of the voice emotion prediction modeling device 240C and the text emotion prediction modeling device 240D may generate a valence prediction value and an arousal prediction value for 6 individual emotions. At this time, the valence prediction value and the arousal prediction value for each individual emotion may be paired with each other. Thus, in this example, each of the voice emotion prediction modeling device 240C and the text emotion prediction modeling device 240D may generate total 6 pairs of predicted values.

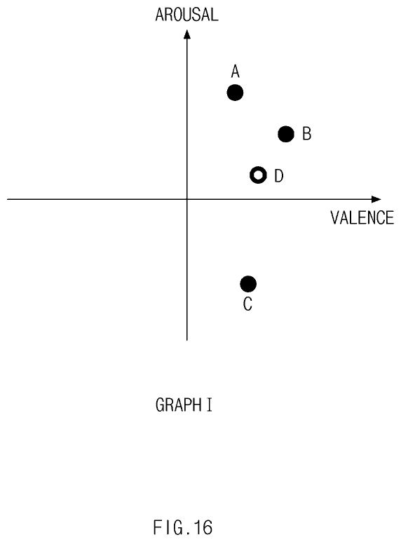

The individual emotion extraction device 240 may transmit pairs of valence and arousal prediction values generated from the modeling devices 240A to 240D to a final emotion determination device 250. A description hereinbelow is provided with reference to FIGS. 16 to 18. However, the final emotion determination device 250 may analyze the received predicted values in a 2D space and may primarily extract an average emotion value in an area with the highest density as the final representative emotion.

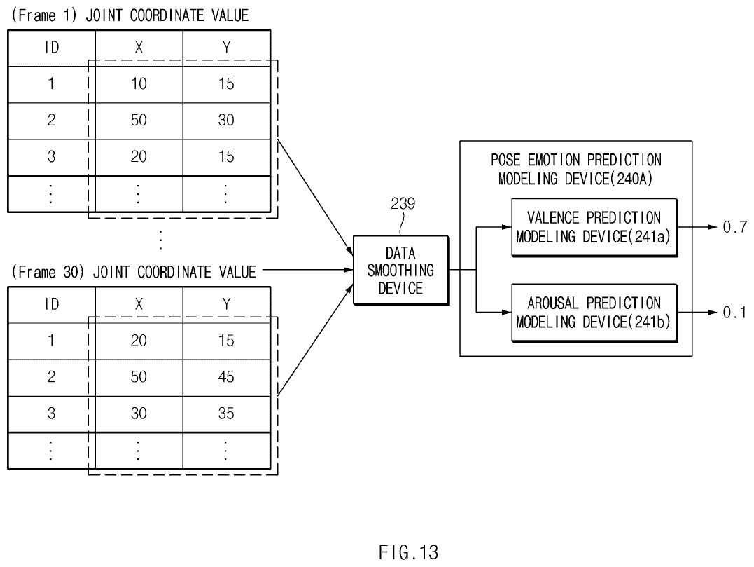

FIG. 13 is a drawing for describing an operation of predicting valence and arousal in a pose emotion prediction modeling device of an individual emotion extraction device according to an example embodiment of the present disclosure.

Referring to FIG. 13, joint coordinate values may be input to a pose emotion prediction modeling device 240A via a data smoothing device 239. The data smoothing device 239 may be included in a preprocessing device 230 and may be disposed between the preprocessing device 230 and an individual emotion extraction device 240. Because a video image in which a user 100-1 gives an answer is captured for each frame by a camera frame capture device 231 and RGB conversion is then performed for a raw image on a frame-by-frame basis by an RGB conversion device 232, a joint coordinate value generated by a pose landmark extraction device 233 may correspond to an image on a frame-by-frame basis. A single frame may be data obtained in a relatively short time, which may not be useful to train the pose emotion prediction modeling device 240A.

Thus, in some embodiments, frames of the image in which the user 100-1 gives an answer may be accumulated and respective joint coordinate values of the accumulated images on a frame-by-frame basis may be smoothed and provided to the pose emotion prediction modeling device 240A. At this time, the number of accumulated frames may be set by the user 100-1.

A left table of FIG. 13 illustratively indicates joint coordinate values of frames 1 to 30. For example, the joint coordinate value of frame 1 and the joint coordinate value of frame 30 may have a list form as follows.

### Joint Coordinate Value of Frame 1

1: (10, 15), 2: (50, 30), 3: (20, 15), . . .

### Joint Coordinate Value of Frame 30

1: (20, 15), 2: (50, 45), 3: (30, 35), . . .

The data smoothing device 239 may sort pieces of X-axis data of frames 1 to 30 and may smooth joint coordinate values of frames 1 to 30 in such a manner as to arrange pieces of Y-axis data. For example, the result of smoothing the joint coordinate values of frames 1 to 30 in the data smoothing device 239 in the example of the joint coordinate values of frames 1 to 30 of FIG. 11 is as follows.
[10, 50, 20, . . . , 20, 50, 30, . . . , 15, 30, 15, . . . , 15, 45, 35, . . . ]

The data smoothing device 239 may transmit the smoothed joint coordinate values of frames 1 to 30 to the pose emotion prediction modeling device 240A. The smoothed joint coordinate values may be input to each of a valence prediction modeling device 241a and an arousal prediction modeling device 241b. The valence prediction modeling device 241a and the arousal prediction modeling device 241b may independently predict valence and arousal, respectively. In some example embodiments, learning models used by the valence prediction modeling device 241a and the arousal prediction modeling device 241b may be models specialized for processing time-series data, such as RNN, LSTM, and BiLSTM, but is not limited thereto.

The valence prediction modeling device 241a and the arousal prediction modeling device 241b may predict valence and arousal of the emotion of a user 100-1, which correspond to the received smoothed joint coordinate values, to generate valence and arousal prediction values. At this time, each of the predicted values generated by the valence prediction modeling device 241a and the arousal prediction modeling device 241b may be a value between −1 and +1.

For example, assuming that Q frames are accumulated and used to train the pose emotion prediction modeling device 240A to analyze a pose of the user 100-1 and the number of joint IDs of the user 100-1 is R (here, Q or R is a natural number of 1 or more), because the number of models included in the pose emotion prediction modeling device 240A is 2, the size of a vector input to the pose emotion prediction modeling device 240A may be 2*Q*R.

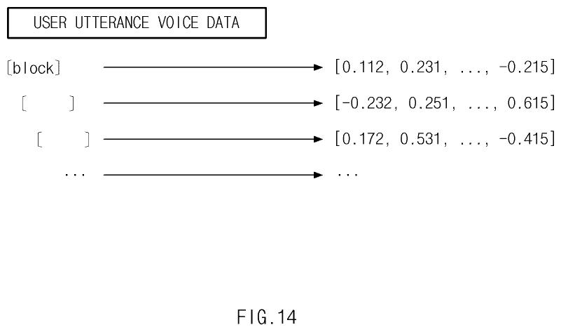

FIG. 14 is a drawing for describing an operation of extracting a frequency feature value in an MFCC voice feature extraction device according to an example embodiment of the present disclosure.

Referring to FIG. 14, when processing voice data of a user 100-1, which is received from an buffer 235, via MFCC preprocessing, an MFCC voice feature extraction device 236 (shown in FIG. 9) may process the voice data in units of a specific time (hereinafter referred to as a “block”) to obtain S coefficient values (here, S is a natural number of 1 or more). The MFCC voice feature extraction device 236 may process voice data of the user 100-1 for one block, may then shift the voice data after a certain time, and may process voice data of the user 100-1 for a next block to obtain S other coefficient values. For example, assuming that the block is 30 ms and the shifted time is 15 ms, the MFCC voice feature extraction device 236 may process voice data of the user 100-1 in units of 30 ms to obtain S coefficient values, may shift the voice data to a time of 15 ms, and may process next voice data of user 100-1 in units of next 30 ms.

According to an example embodiment, when it is not easy to extract the emotion of the user 100-1 from voice data of the user 100-1 in units of 30 ms, a data smoothing device 239 may accumulate and smooth coefficient values of a plurality of blocks and may transmit the smoothed data as an input value of a voice emotion prediction modeling device 240C. A valence prediction modeling device 243a and an arousal prediction modeling device 243b of the voice emotion prediction modeling device 240C may predict valence and arousal of the emotion of the user 100-1 using the smoothed data.

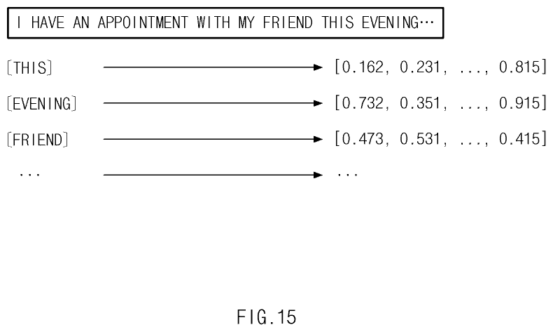

FIG. 15 is a drawing for describing an operation of extracting a word vector value in a word embedding modeling device according to an example embodiment of the present disclosure.

Referring to FIG. 15, a word embedding modeling device 238 (shown in FIG. 9) may receive text data from an STT conversion device 237, may extract a vector for each word for the received text data, and may provide an accumulated value for it as an input value of a text emotion prediction modeling device 240D. At this time, extracting the vector for each word for the text data may refer to a process of excluding a postposition. Furthermore, extracting a vector for each word from an utterance (e.g., sentence) of a user 100-1 may be on the basis of between an utterance starting point and a time point when a pause occurs.

For example, when the utterance (e.g., sentence) of the user 100-1 is “I have an appointment with my friend this evening, so I'm going out,” is uttered, the words, “this” and “evening,” may be extracted from the phrase, “this evening,” and may be converted into T vectors (here, T is a natural number). Thus, a total of 2*T word vector values may be input to the text emotion prediction modeling device 240D. A valence prediction modeling device 244a and an arousal prediction modeling device 244b of the text emotion prediction modeling device 240D may predict valence and arousal of the emotion of the user 100-1 using the received 2*T word vector values.

Next, for example, when the text data, “have an appointment with my friend”, which is a next utterance of the user 100-1 is input a word embedding modeling device 238, a total of 5 words, “today,” “evening,” “friend,” “appointment,” and “have,” including the phrase, “this evening,” which is previously input, may be extracted. Thus, a total of 5*T word vector values may be input to the text emotion prediction modeling device 240D. The valence prediction modeling device 244a and the arousal prediction modeling device 244b of the text emotion prediction modeling device 240D may predict valence and arousal of the emotion of the user 100-1 using the received 5*T word vector values.

FIGS. 16 to 19 are drawings for describing a method for primarily extracting a final emotion in a final emotion determination device according to an example embodiment of the present disclosure. Hereinafter, a description of a method for primarily extracting a final representative emotion of a user 100-1 in a final emotion determination device 250 together with reference to FIG. 2 is provided.

Valence prediction values and arousal prediction values generated by an individual emotion extraction device 240 may be represented in a 2D emotion space as shown in FIG. 11. However, periods when modeling devices 240A to 240D of the individual emotion extraction device 240 predict individual emotions of the user 100-1 during an answer time of the user 100-1 to a question of a robot 200-1 may be different from each other. Thus, the number of pairs of a valence prediction value and an arousal prediction value of each of the individual emotions of the user 100-1 may differ. Thus, there is a need for a process of extracting a final representative emotion for the answer of the user 100-1 to the question of the robot 200-1.

At this time, when the final emotion determination device 250 determines an average value of valence and an average value of arousal of each of the individual emotions as a final representative emotion, an ambiguous emotion different from a real emotion of the user 100-1 may be determined as the emotion of the user 100-1.

For example, referring to FIG. 16, each of coordinates A, B, and C on graph I indicates a pair of a valence prediction value and an arousal prediction value for an individual emotion of the user 100-1. For example, coordinates A may be valence and arousal prediction values generated by the pose emotion prediction modeling device 240A, coordinates B may be valence and arousal prediction values generated by the expression emotion prediction modeling device 240B, and coordinates C may be valence and arousal prediction values generated by the voice emotion prediction modeling device 240C. However, an example embodiment is not limited thereto. A coordinate value of any one of coordinates A, B, and C may be valence and arousal prediction values generated by the text emotion prediction modeling device 240D. Coordinates D may have a coordinate value corresponding to an average value of coordinates A, coordinates B, and coordinates C.

For example, when the emotion of the user 100-1, which is extracted from the valence and arousal prediction values of the pose emotion prediction modeling device 240A and the expression emotion prediction modeling device 240B, is happiness, but the emotion of the user 100-1, which is extracted from the valence and arousal prediction values of the voice emotion prediction modeling device 240C, is calmness, an ambiguous emotion, such as neutral, may be determined as a final representative emotion of the user 100-1. As such, when the final emotion is determined using an average value of the individual emotions of the user 100-1, the ambiguous emotion different from the real emotion of the user 100-1 may be determined as the emotion of the user 100-1.

Referring to FIGS. 2, 17, and 18, the final emotion determination device 250 may apply a Gaussian mixture model to a valence prediction value and an arousal prediction value for each (e.g., piece of) data indicating the emotion of the user 100-1, which are received from the individual emotion extraction device 240, to primarily extract a final representative emotion of the user 100-1. The Gaussian mixture model is an algorithm for mixing several Gaussian normal distributions and approximating the distribution of complex data. When applying the Gaussian mixture model, the final emotion determination device 250 may normalize a valence prediction value and an arousal prediction value for each piece of data indicating the emotion of the user 100-1, which are scattered in a 2D space and may estimate a point with the highest density.

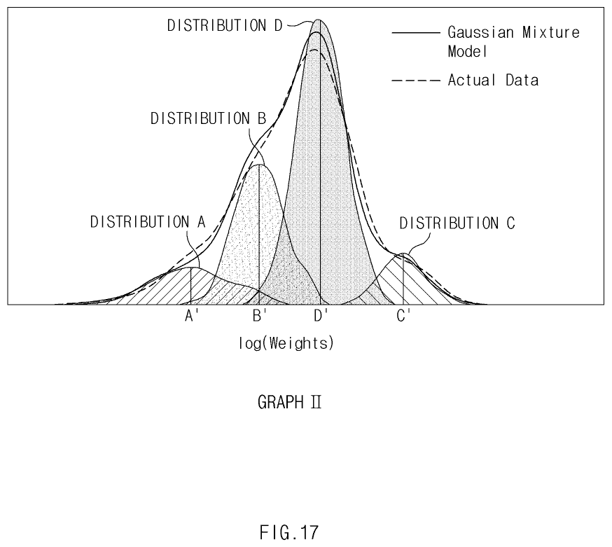

According to the present disclosure, the final emotion determination device 250 may generate Gaussian normal distributions by the number of pieces of individual emotion data indicating the emotion of the user 100-1 using the Gaussian mixture model and may extract a median value with the highest density in each distribution as a representative emotion of the user 100-1. For example, because the number of individual emotion prediction modeling devices of the user 100-1, which are included in the individual emotion extraction device 240, is 4, 4 Gaussian normal distribution curves may be generated as shown in FIG. 17 and a median value of a place with the highest data density on each normal distribution curve may be primarily extracted as a final representative emotion of the user 100-1.

For example, distribution A of graph II may correspond to valence and arousal prediction values generated by the pose emotion prediction modeling device 240A, distribution B may correspond to valence and arousal prediction values generated by the expression emotion prediction modeling device 240B, and distribution C may correspond to valence and arousal prediction values generated by the voice emotion prediction modeling device 240C. However, an example embodiment is not limited thereto. Any one of distributions A, B, and C may correspond to valence and arousal prediction values generated by the text emotion prediction modeling device 240D. A median value of a place with the highest data density in distribution A may be A′, a median value of a place with the highest data density in distribution B may be B′, a median value of a place with the highest data density in distribution C may be C′, and a median value of a place with the highest data density in distribution D may be D′. At this time, the final emotion determination device 250 may primarily extract D′ corresponding to the median value of the place with the highest data density on each normal distribution curve as a final representative emotion of the user 100-1. As such, the final emotion determination device 250 may determine the final representative emotion of the user 100-1 using the Gaussian mixture model, such that a dominant individual emotion of the user 100-1 may be adopted as a representative emotion.

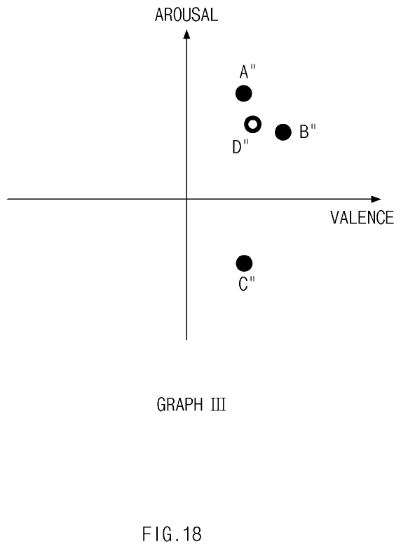

Next, referring to FIG. 18, coordinate values of coordinates A″, B″, and C″ of graph III may be the same as coordinate values of coordinates A, B, and C of graph I. Furthermore, the coordinate value of coordinates D″ of graph III may correspond to a median value of a place with the highest data density when the final emotion determination device 250 applies a Gaussian mixture model to a valence prediction value and an arousal prediction value for each of pieces of data indicating the emotion of the user 100-1. Unlike coordinates D of graph I of FIG. 16, because coordinates D″ of graph III correspond to an average value of an area where the predicted density of the emotion of the user 100-1 is high, the emotion of the user 100-1 may be more accurately predicted. The determination of whether a value is considered “high” may be made based on a predefined threshold.

FIG. 19 is a drawing for describing a process of primarily extracting a representative emotion of a user 100-1 in an emotion-based data analysis system for user emotion monitoring according to an example embodiment of the present disclosure.

Referring to FIG. 19, a robot 200-1 may ask the user 100-1 a plurality of (e.g., 10) questions. The user 100-1 may give an answer to each of the questions of the robot 200-1. A preprocessing device 230, an individual emotion extraction device 240, and a final emotion determination device 250 included in the robot 200-1 may preprocess answer data of the user 100-1 and may primarily extract a final representative emotion of the user 100-1 from the preprocessed data. The primarily extracted final representative emotion of the user 100-1 may be represented as valence and arousal and may be independently represented in a 2D emotion space as shown in graphs shown on the left of FIG. 19.

A user emotion pattern analysis device 260 may receive the primarily extracted final representative emotion from the final emotion determination device 250 and may compare the received primarily extracted final representative emotion with a healthy person's emotion pattern model 212-1 stored in data storage 210. At this time, the user emotion pattern analysis device 260 may secondarily extract a final representative emotion in which a difference with the healthy person's emotion pattern model 212-1 is greater than or equal to a predetermined value among the primarily extracted final representative emotions and may store answer data (i.e., abnormal data) of the user 100-1, which corresponds to it, in an abnormal data storage server device 310.

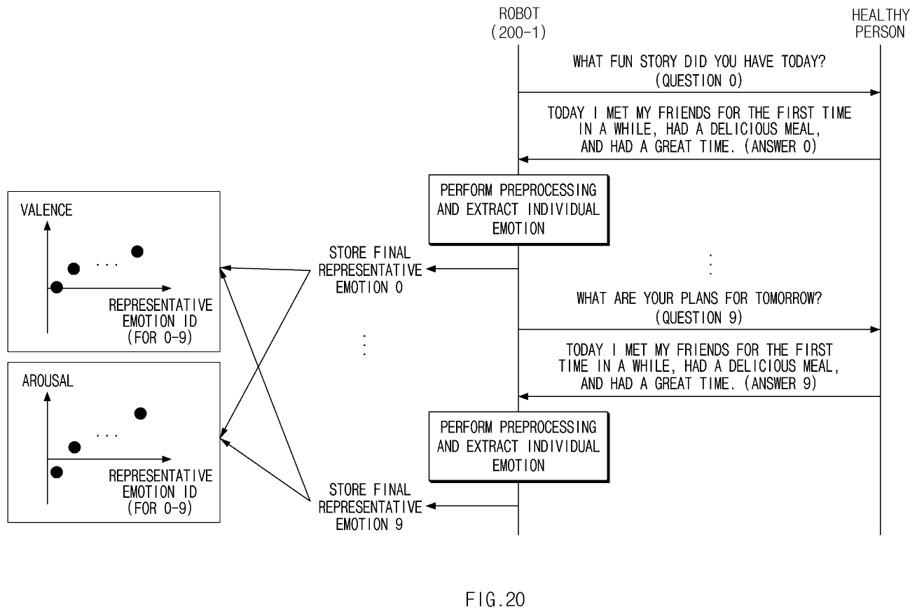

FIG. 20 is a drawing for describing a healthy person's emotion pattern model stored in data storage according to an example embodiment of the present disclosure.

Referring to FIG. 20, a healthy person's emotion pattern model for a predefined question set of a robot 200-1 may be configured in advance via a process similar to the process of primarily extracting the representative emotion of the user 100-1 in the emotion-based data analysis system for user emotion monitoring, which is described with reference to FIG. 19. In detail, the robot 200-1 may ask a healthy person a plurality of (e.g., 10) questions and the healthy person may give an answer to each of the questions of the robot 200-1. The robot 200-1 may obtain answer data of the healthy person via its camera and its microphone, may preprocess the answer data of the healthy person using a preprocessing device 230, an individual emotion extraction device 240, and a final emotion determination device 250, and may primarily extract a final representative emotion of the healthy person with regard to data density and distribution from the preprocessed data. The primarily extracted final representative emotion of the healthy person may be represented as valence and arousal and may be independently represented in a 2D emotion space as shown in graphs shown on the left of FIG. 20. The primarily extracted final representative emotion of the healthy person may correspond to the answer of the healthy person to the question of the robot 200-1. For example, as shown in FIG. 20, when the questions of the robot 200-1 are 10, the primarily extracted final representative emotions of the healthy person may also be 10.

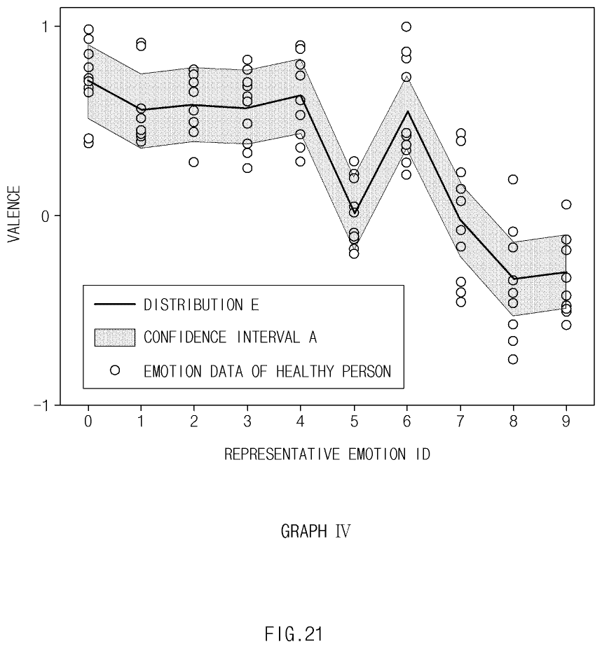

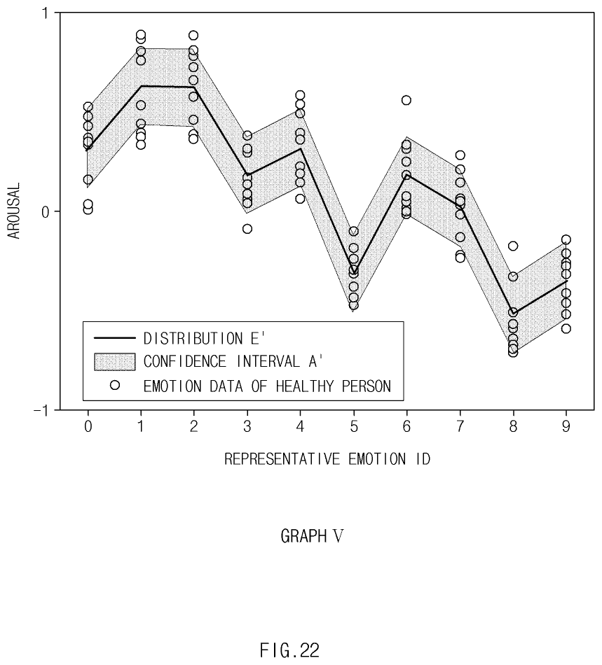

FIGS. 21 and 22 are drawings for describing a method for determining abnormal data using a healthy person's emotion pattern model stored in data storage and secondarily extracting a final emotion of a user in a user emotion pattern analysis device according to an example embodiment of the present disclosure.

Graph IV of FIG. 21 and graph V of FIG. 22 are graphs illustrating data obtained from a plurality of healthy persons via the process described with reference to FIG. 20. The horizontal axis of graph IV of FIG. 21 may indicate a representative emotion ID, and the vertical axis may indicate valence predicted from the answer of the healthy person. The horizontal axis of graph V of FIG. 22 may indicate a representative emotion ID, and the vertical axis may indicate arousal predicted from the answer of the healthy person.

The answer data of the healthy person, which is obtained via the process described with reference to FIG. 20 may be used as data for training and may configure a Gaussian process regression learning model. At this time, Gaussian process regression may learn the distribution of answer data of the healthy person, may determine a confidence interval corresponding to confidence interval A of graph IV and confidence interval A′ of graph V, and may predict an average value of the answer data of the healthy person, which corresponds to distribution E of graph IV and distribution E′ of graph V. At this time, the width of the confidence interval may decrease, when representative emotions of the plurality of healthy persons for the same question are similarly predicted, and the width of the confidence interval may increase, when the representative emotions of the plurality of healthy persons for the same question are differently predicted.

When the final representative emotion of the user 100-1, which is primarily extracted via the process described with reference to FIG. 19, is out of the confidence interval, a user emotion pattern analysis device 260 may determine the final representative emotion as abnormal data.

For example, referring to graph IV, when valence of the final representative emotion of the user 100-1, which is primarily extracted by the final emotion determination device 250 for an 0th question of the robot 200-1, which corresponds to a 0th representative emotion ID, is −0.5, because the valence is out of confidence interval A, the user emotion pattern analysis device 260 may determine the primarily extracted final representative emotion of the user 100-1 as abnormal data.

As such, the user emotion pattern analysis device 260 may compare the final representative emotion primarily extracted by the final emotion determination device 250 with a healthy person's emotion pattern stored in data storage 210 and may secondarily extract a final representative emotion in which a difference with the healthy person's emotion pattern is greater than or equal to a predetermined value to determine the final representative emotion as abnormal data.

According to the present disclosure, the user emotion pattern analysis device 260 may extract abnormal data among emotion patterns of the user using the Gaussian process regression model to (e.g., quickly) extract the abnormal data without (e.g., the necessity) of analyzing previous data whenever data is input.

FIG. 23 is a drawing for describing an example of a computing system associated with an emotion-based data analysis system for user emotion monitoring according to an embodiment of the present disclosure.

Referring to FIG. 23, a computing system 900 may include at least one processor 910, a memory 930, a user interface input device 940, a user interface output device 950, a storage 960, and a network interface 970, which are connected with each other via a bus 920.

The processor 910 may be a central processing unit (CPU) or a semiconductor device that processes (or executes) at least one instruction stored in the memory 930 and/or the storage 960. The memory 930 and the storage 960 may include various types of volatile or non-volatile storage media. For example, the memory 930 may include a read only memory (ROM) 931 and a random access memory (RAM) 932.

Accordingly, the operations of the method or algorithm described in connection with the embodiments disclosed in the specification may be directly implemented with a hardware module, a software module, or a combination of the hardware module and the software module, which is executed by the processor 910. The software module may reside on a storage medium (i.e., the memory 930 and/or the storage module 960) such as a RAM, a flash memory, a ROM, an EPROM, an EEPROM, a register, a hard disc, a removable disk, and a CD-ROM.

The exemplary storage medium may be coupled to the processor 910. The processor 910 may read out information from the storage medium and may write information in the storage medium. Alternatively, the storage medium may be integrated with the processor 910. The processor and the storage medium may reside in an application specific integrated circuit (ASIC). The ASIC may reside within a user terminal. In another case, the processor and the storage medium may reside in the user terminal as separate components.

The multimodal data may be synthesized to predict the emotion of the user, thus increasing the accuracy of predicting the emotion of the user.

Furthermore, tan emotion pattern of a healthy person may be compared with an emotion pattern of a user to upload (e.g., only) abnormal data that a specialist may use (e.g., need) to observe to a server for monitoring by the specialist, thus increasing the efficiency of consultation data monitoring of the specialist.

Furthermore, distribution and density of data for the predicted representative emotion of the user may be considered to determine an average value of the emotion in an area where the density of the predicted emotion is high as a final representative emotion, thus increasing the accuracy of user emotion prediction.

Furthermore, a confidence interval in the data distribution of the healthy person may be determined and (e.g., only) data included in the confidence interval may be regarded as normal data, thus increasing the accuracy of user emotion prediction.

In addition, various impacts (e.g., effects) ascertained directly or indirectly through the present disclosure may be provided.

Although the present disclosure has been described with reference to example embodiments and the accompanying drawings, the present disclosure is not limited thereto, but may be variously modified and altered without departing from the spirit and scope of the present disclosure, including the claims.

Therefore, example embodiments of the present disclosure are not intended to limit the present disclosure, but may be provided for illustrative purposes. The scope of the present disclosure should be construed on the basis of the accompanying claims, and the technical ideas within the scope equivalent or substantially equivalent to the claims should be included in the scope of the present disclosure.

What is claimed is:

1. An emotion-based data analysis system for user emotion monitoring, the emotion-based data analysis system comprising:
data storage configured to store a predefined question set and an emotion pattern of a healthy person corresponding to the predefined question set;
a scenario controller configured to sequentially execute questions of the predefined question set and control an answer acquisition start time and an answer acquisition end time;
a preprocessing device configured to obtain data indicating an emotion of a user to generate preprocessed data, in response to a user answer acquisition start signal received from the scenario controller;
an individual emotion extraction device configured to predict valence and arousal for the data indicating the emotion of the user based on the preprocessed data received from the preprocessing device;
a final emotion determination device configured to analyze predicted values of the valence and the arousal for the data indicating the emotion of the user, the predicted values received from the individual emotion extraction device, in a two-dimensional (2D) space to calculate an average emotion value of an area with highest density and primarily extract the calculated average emotion value as a final representative emotion;
a user emotion pattern analysis device configured to compare the primarily extracted final representative emotion with the emotion pattern of the healthy person stored in the data storage and secondarily extract a final representative emotion in which a difference with the healthy person's emotion pattern is greater than or equal to a predetermined value; and
an abnormal data storage server device configured to receive and store the secondarily extracted final representative emotion from the emotion pattern analysis device.

2. The emotion-based data analysis system of claim 1, wherein the data indicating the emotion of the user include one or more of pose data, expression data, voice data, or text data of the user, and wherein at least one of the scenario controller, the preprocessing device, the individual emotion extraction device, the final emotion determination device, the user emotion pattern analysis device, or the abnormal data storage server device includes a memory storing computer-executable instructions and at least one processor configured to access the memory and execute the computer-executable instructions.

3. The emotion-based data analysis system of claim 1, wherein the predefined question set stored in the data storage includes a plurality of predefined question sets, and
wherein the scenario controller is configured to:
select a question set from the plurality of predefined question sets stored in the data storage; and
sequentially deliver questions of the selected question set to the user.

4. The emotion-based data analysis system of claim 1, wherein the predefined question set includes an Nth question and an (N+1)th question, and
wherein the scenario controller is configured to:
execute the Nth question; and
execute the (N+1)th question, in response to a lack of text conversion corresponding to the Nth question during a predetermined time.

5. The emotion-based data analysis system of claim 1, further comprising:
a camera and a microphone configured to obtain and provide the data indicating the emotion of the user to the preprocessing device, in response to the preprocessing device receiving the user answer acquisition start signal.

6. The emotion-based data analysis system of claim 5, wherein the preprocessing device is configured to:
perform RGB conversion of the data indicating the emotion of the user, the data received from the camera, on a frame-by-frame basis; and
extract landmarks of a pose and an expression of the user from RGB data converted on the frame-by-frame basis to generate the preprocessed data.

7. The emotion-based data analysis system of claim 6, wherein the extracted landmark of the pose of the user includes at least one pair of a joint ID of the user or a 2D coordinate value corresponding to the joint ID of the user.

8. The emotion-based data analysis system of claim 6, wherein the extracted landmark of the expression of the user includes at least one pair of a feature point ID of a face of the user or a 2D coordinate value corresponding to the feature point ID of the face of the user.

9. The emotion-based data analysis system of claim 5, wherein the preprocessing device is configured to:
store the data, received from the microphone, indicating the emotion of the user in a buffer; and
extract a voice feature of voice data stored in the buffer via a Mel-frequency cepstral coefficient (MFCC) to generate the preprocessed data.

10. The emotion-based data analysis system of claim 5, wherein the preprocessing device is configured to:
store the data, received from the microphone, indicating the emotion of the user in a buffer;
convert voice data stored in the buffer into text data; and
convert the text data into a computer-readable vector via a word embedding model to generate the preprocessed data.

11. An operating method of an emotion-based data analysis system for user emotion monitoring, the operating method comprising:
storing, by data storage, a predefined question set and a healthy person's emotion pattern corresponding to the predefined question set;
executing, by a scenario controller, a plurality of questions of the predefined question set;
controlling, by the scenario controller, an answer acquisition start time and an answer acquisition end time for a user for the questions;
receiving, by a preprocessing device, a user answer acquisition start signal from the scenario controller;
obtaining, by the preprocessing device, data indicating an emotion of the user to generate preprocessed data, in response to receiving the user answer acquisition start signal;
receiving, by an individual emotion extraction device, the preprocessed data from the preprocessing device;
predicting, by the individual emotion extraction device, valence and arousal for the data indicating the emotion of the user;
receiving, by a final emotion determination device, predicted values of the valence and the arousal for the data indicating the emotion of the user from the individual emotion extraction device;
analyzing, by the final emotion determination device, the predicted values of the valence and the arousal for the data indicating the emotion of the user, in a two-dimensional (2D) space to calculate an average emotion value of an area with highest density;
primarily extracting, by the final emotion determination device, the calculated average emotion value as a final representative emotion;
comparing, by a user emotion pattern analysis device, the primarily extracted final representative emotion with the healthy person's emotion pattern stored in the data storage and secondarily extracting, by the user emotion pattern analysis device, a final representative emotion in which a difference with the healthy person's emotion pattern is greater than or equal to a predetermined value;
receiving and storing, by an abnormal data storage server device, the secondarily extracted final representative emotion from the user emotion pattern analysis device; and
monitoring, by a specialist, the secondarily extracted final representative emotion stored on the abnormal data storage server device to provide a treatment plan to the user.

12. The operating method of claim 11, wherein the data indicating the emotion of the user includes at least one of pose data, expression data, voice data, or text data of the user.

13. The operating method of claim 11, wherein the predefined question set stored in the data storage includes a plurality of predefined question sets, and
wherein the executing of the questions of the predefined question set by the scenario controller includes:
selecting, by the scenario controller, one predefined question set from the plurality of predefined question sets; and
delivering, by the scenario controller, questions of the selected question set to the user.

14. The operating method of claim 11, wherein the predefined question set includes an Nth question and an (N+1)th question subsequent to the Nth question, and
wherein the executing of the questions of the predefined question set by the scenario controller includes:
executing, by the scenario controller, the Nth question; and
executing, by the scenario controller, the (N+1)th question, in response to the lack of text conversion corresponding to the Nth question during a predetermined time.

15. The operating method of claim 11, further comprising:
obtaining and providing, by a camera and a microphone, the data indicating the emotion of the user to the preprocessing device, in response to that the preprocessing device receives the user answer acquisition start signal.

16. The operating method of claim 15, wherein the generating of the preprocessed data by the preprocessing device includes:
performing RGB conversion of the data indicating the emotion of the user, the data being received from the camera, on a frame-by-frame basis; and
extracting landmarks of a pose and an expression of the user from RGB data converted on the frame-by-frame basis to generate the preprocessed data.

17. The operating method of claim 16, wherein the extracted landmark of the pose of the user includes at least one pair of a joint ID of the user and a 2D coordinate value corresponding to the joint ID of the user.

18. The operating method of claim 16, wherein the extracted landmark of the expression of the user includes at least one pair of a feature point ID of a face of the user and a 2D coordinate value corresponding to the feature point ID of the face of the user.

19. The operating method of claim 15, wherein the generating of the preprocessed data by the preprocessing device includes:
storing the data indicating the emotion of the user, the data received from the microphone, in a buffer; and
extracting a voice feature of voice data stored in the buffer via a Mel-frequency cepstral coefficient (MFCC) to generate the preprocessed data.

20. The operating method of claim 15, wherein the generating of the preprocessed data by the preprocessing device includes:
storing the data indicating the emotion of the user, the data received from the microphone, in a buffer;
converting voice data stored in the buffer into text data; and
converting the text data into a computer-readable vector via a word embedding model to generate the preprocessed data.
